(19) United States
(12) Patent Application Publication
(10) Pub. No.: US 2025/0263539 A1
(43) Pub. Date: Aug. 21, 2025

(54) LOW MOLECULAR WEIGHT LOW TG HYDROCARBON RESINS, RUBBER COMPOSITIONS, AND TIRE COMPOSITIONS

(71) Applicant: Resin Solutions, LLC, Exton, PA (US)

(72) Inventors: Steven Hennng, Downington, PA (US); Robert Adams, Brookhaven, PA (US); Jacob Gregory, West Chester, PA (US)

(73) Assignee: Resin Solutions, LLC, Exton, PA (US)

(21) Appl. No.: 18/418,042

(22) Filed: Feb. 19, 2024

(51) Int. Cl. C08L 9/00; B60C 1/00; C07C 2/38; C08K 3/36; C08K 9/06

(52) U.S. Cl. CPC C08L 9/00; B60C 1/0016; C07C 2/38; C08K 3/36; C08K 9/06; C08L 2207/324

(57) ABSTRACT

A curable rubber composition is provided. The composition includes: a) a liquid oligomer having a number average molecular weight Mn 600 g/mol or less, and a glass transition temperature Tg of −10° C. or less as measured by differential scanning calorimetry DSC, including, as polymerized monomers, at least one diene, at least one branched mono-olefin, and optionally at least one vinyl aromatic monomer; b) at least one polymeric diene-based elastomer having a number average molecular weight of 100,000 g/mol or higher; c) at least one filler comprising at least one of silica, carbon black, or a combination thereof and; d) optionally a curative system capable of curing the curable rubber composition when heated. The cured rubber composition has good traction and low rolling resistance when used as a tire tread.

### FIELD

The invention relates to curable rubber compositions based on diene elastomers which are useful in the manufacture of tire treads.

### BACKGROUND

Tire tread compositions require a combination of low rolling resistance and good traction. It is recognized that the dynamics of these two properties are in opposition. For example, the physical properties necessary to maintain good wet and dry adhesion to the road for safety, and those needed for low rolling resistance for improved fuel economy will often suffer at the expense of reducing one while improving the other. In particular, a high loss modulus (viscous component) is associated with good wet and dry adherence to roadways and good braking, but a high storage modulus (elastic component) is associated with lower (good) rolling resistance. These two properties typically oppose each other, i.e. if one is high, the other tends to be low. Due to current needs for reduced energy consumption, curable tire tread compositions that provide similar or even improved traction, while at the same time providing reduced rolling resistance compared to present technologies are highly desirable.

### SUMMARY OF THE INVENTION

The inventors have found that curable diene-based elastomer (rubber) compositions including certain low molecular weight diene oligomers unexpectedly provide reduced rolling resistance, but maintain excellent traction compared to state of the art such additives in tire tread compositions.

According to one aspect of the invention, a curable rubber composition is provided. The composition includes:
a) a liquid oligomer having a number average molecular weight Mn 600 g/mol or less, and a glass transition temperature Tg of −10° C. or less as measured by differential scanning calorimetry DSC, including, as polymerized monomers, at least one diene, at least one branched mono-olefin, and optionally at least one vinyl aromatic monomer;
b) at least one polymeric diene-based elastomer having a number average molecular weight of 100,000 g/mol or higher;
c) at least one filler comprising at least one of silica, carbon black, or a combination thereof and;
d) optionally a curative system capable of curing the curable rubber composition when heated.

According to another aspect of the invention, method of preparing a liquid oligomer is also provided. The method includes the following steps.

Combining: at least one diene, at least one branched mono-olefin, optionally at least one vinyl aromatic monomer, and at least one initiator including a Lewis acid, to form a reaction mixture. Polymerizing the reaction mixture at a temperature and time effective to form the liquid oligomer. The liquid oligomer thus formed includes, as polymerized monomers, the at least one diene, the at least one branched mono-olefin, and optionally the at least one vinyl aromatic monomer. The liquid oligomer has a number average molecular weight Mn of 600 g/mol or less, and a glass transition temperature Tg of −10° C. or less as measured by differential scanning calorimetry (DSC).

A cured rubber composition including the liquid oligomer is also provided, as well as a tire tread including the cured rubber composition.

### BRIEF DESCRIPTION OF THE DRAWINGS

FIG. 1 shows the tan delta at 0° C., which correlates to traction, plotted as a function of tan delta at 60° C., which correlates to rolling resistance.

### DETAILED DESCRIPTION

Improved rubber compositions may be produced using aspects of the present invention. Applicants have discovered that a balance of properties of good traction together with low rolling resistance may be achieved by the addition of certain liquid low number average molecular weight Mn diene-based oligomers. Thus, reduced fuel consumption may be obtained by developing tires having a very low rolling resistance combined with excellent grip properties and handling behavior, thereby promoting significant cost and environmental benefits. In particular, it was discovered that while tire traction indicators can primarily be influenced by the Tg of the liquid oligomer added to the rubber tread compound, the rolling resistance or fuel economy indicators of a cured diene-based rubber composition are correlated directly with molecular weight of the liquid oligomer that is added to the diene-based rubber composition. In particular, the largest improvement in this indicator is for liquid diene-based oligomers with number average molecular weight Mn<600 g/mol. According to some embodiments, these low Mn's can most efficiently be achieved by polymerizing the monomers with Lewis acid initiators, such as BF₃ or BF₃ etherate initiators. According to some embodiments, the low molar mass liquid oligomers may advantageously include C5 dienes and mono-olefins, especially branched mono-olefins. According to some embodiments the oligomers may further include an aromatic monomer, such as alpha-methylstyrene or styrene aromatic comonomers.

As used herein, the term “oligomer” refers to a polymer repeating unit having 100 monomers or less.

As used herein the term “liquid” means that the oligomer has a Brookfield viscosity of 400,000 cP (mPa·s) or less as measured using an S25 spindle at 0.8 rpm at 25° C.

As used herein, the term “phr” means parts per hundred parts rubber, in weight.

As illustrated in the Examples, the tan delta at 60° C. is correlated to the rolling resistance, where lower is better and the tan delta at 0° C. is correlated with traction, where higher is better.

### Curable Rubber Composition

Thus, a curable rubber composition is provided. The composition includes:
a) a liquid oligomer having a number average molecular weight Mn 600 g/mol or less, and a glass transition temperature Tg of −10° C. or less as measured by differential scanning calorimetry DSC, including, as polymerized monomers, at least one diene, at least one branched mono-olefin, and optionally at least one vinyl aromatic monomer;
b) at least one polymeric diene-based elastomer having a number average molecular weight of 100,000 g/mol or higher;
c) at least one filler comprising at least one of silica, carbon black, or a combination thereof and;
d) optionally a curative system capable of curing the curable rubber composition when heated.

The curable rubber composition may include from 5 to 40 parts per hundred parts rubber (phr) of the liquid oligomer, by weight of the curable rubber composition. According to some embodiments, the curable rubber composition may include at least 5, 10, 15, 20, 25, 40, 30 or at least 35 phr of the liquid oligomer. According to some embodiments, the curable rubber composition may include at most 40, 35, 30, or at most 25 phr of the liquid oligomer. According to some embodiments, the curable rubber composition may include from 5 to 35, from 5 to 30, from 5 to 25, or from 5 to 20 phr of the liquid oligomer. According to some embodiments, the curable rubber composition may include from 10 to 40, from 15 to 40, or from 20 to 40 phr of the liquid oligomer. According to some embodiments, the liquid rubber composition may include from 10 to 35, or from 15 to 30 phr of the liquid oligomer.

According to some embodiments, the curable rubber composition may include from 50 to 120 phr of the filler. According to some embodiments, between about 80 and 100 parts by weight of reinforcing filler may be used per 100 parts of rubber.

### a) Liquid Oligomer

According to an embodiment the liquid oligomer has a number average molecular weight Mn of 600 g/mol or less. The Mn of the oligomer is measured using gel permeation chromatography (GPC) as described in the Examples. According to an embodiment, the Mn of the liquid oligomer may be from 200 g/mol to 600 g/mol. According to an embodiment, the Mn of the liquid oligomer may be from 200 to 600 g/mol. According to some embodiments, the Mn of the liquid oligomer may be at least 200, 225, 250, 275, 300, 325, 350, 375, 400, 425, 450, 475, or at least 500 g/mol as measured by GPC. According to some embodiments the Mn of the oligomer may be at most 600, 575, 550, 525, 500, 475, 450, 425, 400, 375, 350, or at most 300 g/mol as measured by GPC. According to some embodiments, the Mn of the liquid oligomer may be from 200-600 g/mol, or from 200-575, or from 200-550, or from 200-525, or from 200-500, or from 200-450, or from 200-400 g/mol. According to some embodiments, the Mn of the liquid oligomer may be from 225-600 g/mol, or from 250-600, or from 275-600, or from 300-600, 325-600, or from 350-600, or from 375-600, or from 400-600, or from 425-600, or from 450-600, or from 475-600, or from 500-600 g/mol.

The Tg of the liquid oligomer is measured by differential scanning calorimetry (DSC) as described in the Examples. According to an embodiment, the liquid oligomer has a glass transition temperature Tg of −10° C. or less. According to some embodiments, the Tg of the liquid oligomer may be at most −10, −20, −25, −30, −35, −40, or at most −50° C. According to some embodiments, the Tg of the liquid oligomer is at least −100° C., or at least −90, −80, −70, −60, −50, −40, −30, or at least −20° C. According to an embodiment, the Tg of the liquid oligomer may be from −70 to −20° C. According to some embodiments, the Tg of the liquid oligomer may be from −100° C. to −20°° C., or from −100 to −25° C., or from −100 to −30° C., or from −100 to −35° C., or from −100 to −40° C., or from −100 to −50° C. According to some embodiments, the Tg of the liquid oligomer may be from −90° C. to −20° C., or from −80 to −20° C., or from −70 to −20°° C., or from −60 to −20° C., or from −50 to −20° C., from −40° C. to −20° C., or from −20 to −15° C.

### Dienes in the Liquid Oligomer

According to an embodiment, the at least one diene in the liquid oligomer may include C5 dienes, dimers thereof, or combinations thereof. According to an embodiment, the diene in the liquid oligomer may include at least one of piperylene, pentadiene, isoprene, dicyclopentadiene, or combinations thereof. According to an embodiment, the diene in the liquid oligomer may include at least one of piperylene, isoprene, dicyclopentadiene, cyclopentene, or combinations thereof.

According to an embodiment, the liquid oligomer may include from 5 to 100 wt % of the diene, as a polymerized monomer, by weight of the liquid oligomer. According to some embodiments, the liquid oligomer may include at least 10, 20, 30, 35, 40, 45, 50, 55, 60, 65, 70, 75, 80, 85, 90, 95 or at least 98 wt % or up to 100 wt % of the diene as a polymerized monomer, by weight of the liquid oligomer. According to some embodiments, the liquid monomer may include at most 99, 98, 95, 90, 85, 80, 75, 70, 65, 60, 55, 50, 45, 40, 35, 30, or at most 25 wt % of the diene as a polymerized monomer by weight of the liquid oligomer. According to some embodiments, the liquid oligomer may include from 25 to 60 wt % of at least one C5 diene comprising at least one of piperylene, isoprene, dicyclopentadiene, or combinations thereof. According to some embodiments, the liquid oligomer may include from 25 to 55 wt %, or from 30 wt % to 50 wt %, or from 35-45 wt % of the at least one C5 diene as polymerized monomer, by weight of the liquid oligomer.

### Mono-Olefins

According to an embodiment, the liquid oligomer includes at least one mono-olefin. According to another embodiment, the mono-olefin may be branched or linear. According to yet another embodiment the mono-olefin may be a C2-C8 branched or linear mono-olefin. According to an embodiment, the liquid oligomer may include at least one branched mono-olefin. According to still another an embodiment the branched mono-olefin may be a C4-C6 branched mono-olefin. According to some embodiments, the at least one branched mono-olefin includes at least one of isoamylene, isobutylene, or combinations thereof.

According to some embodiments, the liquid oligomer may include at least 20, 25, 30, 35, 40 or at least 45 wt % of the mono-olefin, as a polymerized monomer, by weight of the liquid oligomer.

According to some embodiments, the liquid oligomer may include at most 50, 45, 35, 30, or at most 25 wt % of the mono-olefin, as a polymerized monomer, by weight of the liquid oligomer.

According to some embodiments, the liquid oligomer may include from 20 to 50 wt % of the mono-olefin, as polymerized monomer, by weight of the liquid oligomer. According to some embodiments, the liquid oligomer may include from 20 to 45, from 20 to 40, from 20 to 35, or from 20 to 30 wt % of the mono-olefin, as polymerized monomer, by weight of the liquid oligomer. According to some embodiments, the liquid oligomer may include from 25 to 50, from 30 to 50, from 35 to 50, from 40 to 50, or from 45 to 50 wt % of the mono-olefin, as polymerized monomer, by weight of the liquid oligomer. According to some embodiments, the liquid oligomer may include from 25 to 45, or from 30 to 45 wt % of the mono-olefin, as polymerized monomer, by weight of the liquid oligomer.

According to some embodiments, the liquid oligomer may include from 20 to 50 wt % of the at least one branched olefin comprising at least one of isoamylene, isobutylene, or combinations thereof. According to some embodiments, the liquid oligomer may include from 20 to 50 wt % of at least one of isoamylene, isobutylene, or combinations thereof, as polymerized monomer, by weight of the liquid oligomer. According to some embodiments, the liquid oligomer may include from 20 to 45, from 20 to 40, from 20 to 35, or from 20 to 30 wt % of at least one of isoamylene, isobutylene, or combinations thereof, as polymerized monomer, by weight of the liquid oligomer. According to some embodiments, the liquid oligomer may include from 25 to 50, from 30 to 50, from 35 to 50, from 40 to 50, or from 45 to 50 wt % of at least one of isoamylene, isobutylene, or combinations thereof, as polymerized monomer, by weight of the liquid oligomer. According to some embodiments, the liquid oligomer may include from 25 to 45, or from 30 to 45 wt % of at least one of isoamylene, isobutylene, or combinations thereof, as polymerized monomer, by weight of the liquid oligomer.

### Vinyl Aromatic Monomers

According to an embodiment, the liquid oligomer may include, as a polymerized monomer, a vinyl aromatic monomer.

According to an embodiment, the liquid oligomer may include from 0 to 50 wt % of the vinyl aromatic monomer, as a polymerized monomer, by weight of the liquid oligomer. According to some embodiments, the liquid oligomer may include at least 1, 5, 10, 15, 20, 25, 30, 35, 40, or at least 45 wt % of the vinyl aromatic monomer, as a polymerized monomer, by weight of the liquid oligomer. According to some embodiments, the liquid oligomer may include at most at most 50 wt % of the vinyl aromatic monomer, as polymerized monomer, by weight of the liquid oligomer. According to an embodiment, the liquid oligomer may include from 40 to 50 wt % of the vinyl aromatic monomer, by weight of the liquid monomer.

According to some embodiments, the vinyl aromatic monomer may include at least one of styrene, alpha-methyl styrene, meta-methyl styrene, para-methyl styrene, vinyltoluene, para-t-butylstyrene, methoxystyrenes, chlorostyrenes, vinylmesitylene, divinylbenzene, vinylnaphthalene, or combinations thereof. According to an embodiment, the vinyl aromatic monomer may be alpha-methyl styrene. According to an embodiment, the liquid oligomer may include from 40 to 50 wt % of alpha-methyl styrene, as a polymerized monomer, by weight of the liquid oligomer.

### Other Monomers in the Liquid Oligomer

According to some embodiments, the liquid oligomer optionally further comprises, as polymerized monomers, at least one of myrcene, farnesene, or combination thereof, by weight of the liquid oligomer.

If present, the liquid oligomer may include from 1-60 wt % of these monomers. According to some embodiments, the liquid oligomer may include from 1 to 50, from 1 to 40 from 1 to 30, from 1 to 20, from 1 to 10, or from 1 to 5 wt % of at least one of myrcene, farnesene, or combination thereof, as polymerized monomers, by weight of the liquid oligomer.

According to an embodiment, the liquid oligomer comprises, as polymerized monomers, by weight of the liquid oligomer:
from 40 to 50 wt % of alpha-methyl styrene;
from 25 to 60 wt % of at least one C5 diene comprising at least one of piperylene, isoprene, dicyclopentadiene, or combinations thereof;
from 20 to 50 wt % of at least one branched olefin comprising at least one of isoamylene, isobutylene, or combinations thereof;
optionally from 0 to 60 wt % at least one of myrcene, farnesene, divinylbenzene, or combinations thereof.
and wherein the liquid oligomer has a Tg from −70 to −10° C. and a number average molecular weight Mn from 300 to 600 g/mol.

The rubber compositions according to embodiments of the present invention may be compounded by conventional means known by those having skill in the art, including a mixer or compounder (such as a Banbury® mixer), mill, extruder, etc. The tires may be built, shaped, molded, and cured by various methods which will also be readily apparent to those having skill in such art.

### b) Polymeric Diene-Based Elastomer

The curable composition includes at least one polymeric diene-based elastomer having a number average molecular weight of 100,000 g/mol or higher.

According to some embodiments, the polymeric diene-based elastomer may include at least one of polybutadienes, polyisoprenes, natural rubber, copolymers of butadiene and vinyl aromatic monomers, poly (styrene-co-butadiene), copolymers of isoprene and vinyl aromatic monomers, styrene-isoprene-butadiene rubber (SIBR), styrene-isoprene rubber (SIR), isoprene-butadiene rubber (IBR), natural rubber, polybutadiene, including cis 1,4-polybutadiene), polyisoprene, including cis-1,4-polyisoprene, ethylene/propylene/diene terpolymers (EPDM), and combinations thereof.

The polymeric diene based elastomer may also be functionalized to interact with the filler through covalent or ionic bonds. The functional group may be located at both or one end of the elastomer chain, added in the middle of the chain through a coupling reaction, or added post-polymerization through a grafting reaction. Typical functional groups include alkoxysilanes and/or amines.

The curable rubber compositions used to prepare tires and other products in accordance with the invention may contain a single diene elastomer or a mixture of several diene elastomers, the diene elastomer(s) possibly being used in association with any type of synthetic elastomer other than a diene elastomer, or even with polymers other than elastomers, for example thermoplastic polymers. The high molecular weight diene-based elastomers may include polybutadienes, polyisoprenes, copolymers of butadiene and vinyl aromatic monomers, copolymers of isoprene and vinyl aromatic monomers, and combinations of two or more such diene elastomers. For example, elastomers that may be used in the present invention include styrene-isoprene-butadiene rubber (SIBR), styrene-isoprene rubber (SIR), isoprene-butadiene rubber (IBR). Natural rubber can also be used in addition to synthetic rubbers which may include neoprene (polychloroprene), polybutadiene (including cis 1,4-polybutadiene), polyisoprene (including cis-1,4-polyisoprene), as well as ethylene/propylene terpolymers, also known as ethylene/propylene/diene monomer (EPDM) neodymium catalyzed butadiene rubber. Additional examples of rubbers which may be used include carboxylated rubbers, as well as silicon-coupled and tin-coupled star-branched polymers.

### Fillers

Examples of reinforcing fillers that may be included in the curable rubber compositions according to certain embodiments of the present invention include pyrogenic silica fillers and precipitated finely-divided silicas typically employed for rubber compounding. The silica filler may be of the type obtained by precipitation from a soluble silicate, such as sodium silicate. For example, silica fillers may be produced according to the method described in U.S. Pat. No. 2,940,830, which is incorporated herein in its entirety for all purposes. The precipitated, hydrated silica pigments may have an SiO₂ content of at least 50% and usually greater than 80% by weight on an anhydrous basis. The silica filler may have an ultimate particle size in the range of from about 50 to 10,000 angstroms, between 50 and 400 or between 100 and 300 angstroms. The silica may have an average ultimate particle size in a range of about 0.01 to 0.05 microns as determined by electron microscope, although the silica particles may even be smaller in size. The Brunauer-Emmett-Teller (“BET”) surface area of the filler as measured using nitrogen gas may be in the range of 40 to 600 square meters per gram, or in the range of 50 to 300 square meters per gram. The BET method of measuring surface area is described in the Journal of the American Chemical Society, Vol. 60, page 304 (1930). The silica also may have a dibutylphthalate (“DBP”) absorption value in a range of about 200 to about 400, or in a range of from about 220 to 300.

Various commercially available silicas and/or carbon blacks may be used as reinforcing fillers in various embodiments of the present invention. Suitable types of carbon black include, but are not limited to, super abrasion furnace, intermediate SAF, high abrasion furnace, easy processing channel, fast extruding furnace, high modulus furnace, semi-reinforcing furnace, fine thermal, and/or medium thermal carbon blacks. For example, silicas commercially available from PPG Industries under the Hi-Sil trademark such as, for example, those with designations 210, 243, etc.; silicas available from Rhone-Poulenc, with designations of Z1165MP and Z165GR and silicas available from Degussa AG with designations VN2 and VN3, etc. may be used. The Rhone-Poulenc Z1165MP silica is an example of a silica, which is reportedly characterized by having a BET surface area of about 160-170, a DBP value of about 250-290, and a substantially spherical shape. Suitable examples of carbon blacks include, but are not limited to, N110, N121, N220, N231, N234, N242, N293, N299, S315, N326, N330, N332, N339, N343, N347, N351, N358, N375, N539, N550, N582, N630, N642, N650, N683, N754, N762, N765, N774, N787, N907, N908, N990 and N991. While carbon black is optional, tire formulations generally include it.

Other types of exemplary reinforcing fillers suitable for use in the present invention include, but are not limited to, aluminas, aluminum hydroxides, carbon blacks modified by silica, and the like. Suitable alumina reinforcing fillers include, for example, highly dispersible alumina having a BET surface area from 30 to 400 m2/g, or between 60 and 250m2/g, an average particle size of at most 500 nm, or an average particle size of at most 200 nm.

The reinforcing filler, in particular the reinforcing inorganic filler, may be in any desired or advantageous physical state such as, for example, a powder, micro-beads, hollow beads, granules, balls, spheres, irregular granules, high aspect particles or the like or a combination of such physical states.

Representative reinforcing fillers may be included in rubber compositions according to various embodiments of the invention in amounts ranging from about 5 to 100 parts by weight of reinforcing fillers per 100 parts by weight of the total rubber composition (phr).

For example, between about 10 and 50 parts by weight of reinforcing filler may be used per 100 parts of rubber.

### Curative System

The curable rubber composition includes a curative system. As is known in the art, the purpose of the curative system is to cure, i.e., crosslink or vulcanize the rubber composition. According to an embodiment, the curative system may include at least one of elemental sulfur, organosulfur compounds, or combinations thereof. According to another embodiment, the curative system may additionally include an accelerator, a curing aid, an activator or a combination thereof. Examples of sulfur vulcanizing agents include, but are not limited to, elemental sulfur (free sulfur) or sulfur donating vulcanizing agents, for example, an amine disulfide, polymeric polysulfide or sulfur olefin adducts. The amount of sulfur vulcanizing agent will vary depending on the type of rubber and particular type of sulfur vulcanizing agent, but generally range from about 0.1 phr to about 5 phr with a or from about 0.5 phr to about 2 phr. An example of an activator includes, but is not limited to, the combination of zinc oxide and stearic acid.

Examples of accelerators include, but are not limited to, amines, guanidines, thioureas, thiols, thiurams, disulfides, thiazoles, sulfenamides, dithiocarbamates, and xanthates. In cases where only a primary accelerator is used, the amounts used may range from about 0.5 to 2.5 phr. In cases where combinations of two or more accelerators are used, the primary accelerator may be used in amounts ranging from 0.5 to 2.0 phr and the secondary accelerator may be used in amounts ranging from about 0.1 to 0.5 phr. Combinations of accelerators have been known to produce a synergistic effect. The primary accelerator may be a sulfenamide. If a secondary accelerator is used, it is may be a guanidine, a dithiocarbamate, and/or a thiuram compound.

According to an embodiment, the curative system may include at least one peroxide or metal oxides. The peroxide vulcanizing agents may be at least one of dicumyl peroxide, di-t-butyl peroxide, t-butylcumyl peroxide, benzoyl peroxide, 1,3-bis(t-butylperoxyisopropyl) benzene, 2,5-dimethyl-2,5-di(t-butylperoxy) hexene-3,2,5-dimethyl-2,5-(benzoylperoxy) hexane, 2,5-dimethyl-2,5-mono(t-butylperoxy) hexane, and the like. These organic peroxides may be used alone or in combination. Vulcanizing co-agents may be added to enhance the degree of vulcanization, to avoid tack wear problems. The vulcanization co-agents may be at least one of triallylisocyanurate (TIAC), triallylcyanurate (TAC), 1,2-polybutadiene, metallic salts of unsaturated carboxylic acids, oximes, guanidine, trimethylolpropane trimethacrylate, ethylene glycol dimethacrylate, N-N′-m-phenylene bismaleimide, sulfur, and the like.

### Additives in the Curable Rubber Composition

The curable rubber composition of claim 1, further comprising at least one silane coupling agent, wherein the silane group is represented by the following formula: —Si(OR)₃, where each R is independently a C1-C6 alkyl group or an aryl group. According to an embodiment, the silane coupling agent may be a bis-silane such as bis[3-(triethoxysilyl)propyl]tetrasulfide

When compounding a rubber composition containing a filler, one generally uses a coupling agent. Silane coupling agents may be used. Such coupling agents, for example, may be premixed or pre-reacted with the filler or added to the rubber mix during the rubber/filler processing or mixing stage. If the coupling agent and filler are added separately to the rubber mix during the rubber/filler mixing or processing stage, it is considered that the coupling agent then combines in situ with the filler. Any coupling agents known to those of skill in the art may be employed in compositions of the present invention. Coupling agents are generally composed of a coupling agent which has a constituent silane component (i.e. moiety) capable of reacting with the silica surface and, also, a constituent component capable of reacting with the rubber. The coupling agent may be capable of reacting with a sulfur-vulcanizable rubber, which contains carbon-to-carbon double bonds, or unsaturation. In this manner, the coupler (coupling agent) may act as a connecting bridge between the silica and the rubber and, thereby, enhance the rubber reinforcement aspect of the silica.

The silane of the coupling agent may form a bond to the silica surface, possibly through hydrolysis, and the rubber reactive component of the coupling agent combines with the rubber itself. Usually the rubber reactive component of the coupler is temperature sensitive and tends to combine with the rubber during the final and higher temperature sulfur vulcanization stage. However, some degree of combination or bonding may occur between the rubber-reactive component of the coupler and the rubber during an initial rubber/silica/coupler mixing stage prior to a subsequent vulcanization stage. Silane coupling agents can be used in rubber mixtures to improve the processability and to bind the silica filler and other optionally present polar fillers to the diene rubber. Here, one or more different silane coupling agents can be used in combination with one another. The rubber mixture can thus contain a mixture of different silanes.

The silane coupling agents may react with the surface silanol groups of the silica filler or other polar groups during the mixing of the rubber or the rubber mixture (in situ). Suitable silane coupling agents can be all the silane coupling agents known to one skilled in the art for use in rubber mixtures. Such coupling agents may be bifunctional organosilanes which have at least one alkoxy, cycloalkoxy or phenoxy group on the silicon atom as the leaving group and which, as other functionality, have a group which, if appropriate, can undergo a chemical reaction with the double bonds of the polymer after cleavage. The last-mentioned group can, for example, be the following chemical groups: SCN, —SH, —NH₂ or —Sₓ— (with x=2 to 8). The following are thus suitable as silane coupling agents, for example, (3-mercaptopropyl)triethoxysilane, (3-thiocyanatopropyl)trimethoxysilane or 3,3′-bis(triethoxysilylpropyl) polysulphides with 2 to 8 sulphur atoms, for example, 3,3′-bis(triethoxysilylpropyl) tetrasulphide (TESPT), the corresponding disulphide (TESPD), or also mixtures of the sulphides with 1 to 8 sulphur atoms with different contents of the various sulphides. TESPT can, for example, also be added as a mixture with an industrial carbon black (trade name X50S® from Evonik). For example, a silane mixture which contains 40 to 100 wt % of disulphides, or 55 to 85 wt % of disulphides, or 60 to 80 wt % of disulphides may be used. Such a mixture is, for example, obtainable under the trade name Si 261® from Evonik which, for example, is described in DE 102006004062, the disclosure of which is incorporated by reference herein in its entirety.

Blocked mercaptosilanes such as, for example, from WO 99/09036, the disclosure of which is incorporated by reference herein in its entirety for all purposes, can also be used as silane coupling agents. Silanes such as are described in WO 2008/083241 A1, WO 2008/083242 A1, WO 2008/083243 083243A1, and WO 2008/083244 A1, the disclosures of all of which are incorporated by reference herein in their entireties, can also be used. Silanes which can be used, for example, are known by the name NXT (e.g., (3-(octanoylthio)-1-propyl)triethoxysilane) in various variants are marketed by the Momentive company, USA, or by the name VP Si 363® marketed by Evonik Industries.

Suitable amounts of silane coupling agents may be from 5 to 12 phr based on the phr of silica filler.

The rubber composition may also contain conventional additives in addition to reinforcing fillers, including other fillers, peptizing agents, pigments (other than reinforcing fillers), stearic acid, accelerators, sulfur vulcanizing agents, antiozonants, swelling agents, antioxidants, antiozonants, synthetic and natural fibers, plasticizers, tackifying agents, processing oils, activators, initiators, plasticizers, waxes, stabilizers, prevulcanization inhibitors, extender oils, and the like.

Examples of antidegradants that may be in a rubber composition according to various embodiments of the present invention include, but are not limited to, monophenols, bisphenols, thiobisphenols, polyphenols, hydroquinone derivatives, phosphites, phosphate blends, thioesters, naphthylamines, diphenol amines as well as other diaryl amine derivatives, para-phenylene diamines, quinolines and blended amines. Antidegradants are generally used in an amount ranging from about 0.1 phr to about 10 phr or from about 2 to 6 phr. Examples of a peptizing agent include, but are not limited to, pentachlorophenol, which may be used in an amount ranging from about 0.1 phr to 0.4 phr, or from about 0.2 to 0.3 phr.

### Applications

A cured rubber composition obtained by curing the curable rubber composition is provided. To cure the curable rubber compositions of the present invention, any of the usual vulcanization or curing processes known in the art may be used such as heating with superheated steam or hot air in a press or mold. Accordingly, the curable rubber composition may be cured by a process comprising heating the curable rubber composition, which may be molded into a desired form, at a temperature and for a time effective to cure the diene elastomer(s). A method of making a tire tread, comprising molding and curing the curable rubber composition is provided.

A tire tread, comprising the cured rubber composition is provided. A tire, comprising the tire tread is also provided. Particular embodiments of the present invention include tires, in particular tire treads, that are intended for passenger-car or light truck tires but the invention is not limited only to such tires. It is noted that the particular embodiments of the tires of the present invention are intended to be fitted on motor vehicles (including passenger vehicles) or non-motor vehicles such as bicycles, motorcycles, racing cars, industrial vehicles such as vans, heavy vehicles such as buses and trucks, off-road vehicles such as agricultural, mining, and construction machinery, aircraft or other transport or handling vehicles.

The curable rubber compositions disclosed herein may be used for various rubber products such as tires, particularly a tread compound, and in other components for tires, industrial rubber products, seals, timing belts, power transmission belting, and other rubber goods. As such, the present invention includes products made from the curable rubber compositions disclosed herein.

### Methods of Preparing the Liquid Oligomer

According to an embodiment, a method of preparing a liquid oligomer is provided. The method includes the following steps.

Combining at least one diene, at least one branched mono-olefin, optionally at least one vinyl aromatic monomer, and at least one initiator including a Lewis acid, to form a reaction mixture.

Polymerizing the reaction mixture at a temperature and time effective to form the liquid oligomer. As discussed above, the liquid oligomer includes, as polymerized monomers, the at least one diene, the at least one branched mono-olefin, and optionally the at least one vinyl aromatic monomer. The liquid oligomer has a number average molecular weight Mn of 600 g/mol or less, and a glass transition temperature Tg of −20° C. or less as measured by differential scanning calorimetry (DSC).

The polymerization may be carried out as a continuous, semi-continuous, semi-batch or batch process. According to an embodiment, the polymerization may include a solvent. Non-limiting examples of suitable solvents are aromatic or aliphatic hydrocarbon solvents, such as, for example, toluene, xylene, or heptane. According to an embodiment, in the polymerization process, the Lewis acid, monomers, and a suitable aromatic or aliphatic hydrocarbon solvent, such as, for example, toluene, xylene, or heptane, are continuously added to a reactor vessel to form the desired liquid. Methods of producing the liquid oligomer according to the present invention include combining the monomer mixture and a solvent, to provide a monomer feed, and polymerizing the monomer feed by combining the monomer feed with a Lewis acid initiator in a vessel. Polymerizing the monomer feed may include continuously, semi-continuously, semi-batch or batch-feeding the monomer feed and the Lewis acid to the vessel. According to an embodiment, the flow rate of monomer feed and Lewis acid is controlled, such that the Lewis acid feed is about 0.01 to 20 wt. % based on the combined mass flow rate of the monomer, or about 0.1 to 5 wt. %, or about 0.1 to 3 wt. %. The reaction temperature of the mixture in the reactor vessel may be maintained at a temperature of from 5 to 45° C. or from 10 to 30° C., or from 20 to 40° C.

A Lewis acid initiator is a compound or ionic species which can accept an electron pair from a donor compound. Non-limiting examples include Friedel Crafts initiators. According to an embodiment, the Lewis acid may include at least one of SnCl₄, AlCl₃, BF₃, or TiCl₄. According to an embodiment, the Lewis acid may include at least one of AlCl₃, BF₃ or BF₃ etherate.

According to an embodiment, the initiator may further include a protic source. A protic source is a species capable of donating a cation, or a hydrogen having a positive charge. Non-limiting examples include water, esters, anhydrides, or alcohols, especially lower aliphatic or branched alcohols such as methanol, ethanol, or isopropyl alcohol. According to an embodiment the protic source may be an alcohol. According to an embodiment the alcohol may be methanol, ethanol, isopropyl alcohol, or a combination thereof.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the composition or process. Additionally, in some embodiments, the invention can be construed as excluding any element or process step not specified herein.

Although the invention is illustrated and described herein with reference to specific embodiments, the invention is not intended to be limited to the details shown. Rather, various modifications may be made in the details within the scope and range of equivalents of the claims and without departing from the invention.

### EXAMPLES

### Methods

Resin Glass Transition Temperature (Tg) was measured using Differential Scanning calorimetry (DSC) on a TA Instruments DSC2500. Resin samples (5-20 mg) were loaded in aluminum hermetically sealed pans, cooled to 50° C. below their expected Tg, and Tg was measured after a temperature ramp of 10° C. per minute.
Viscoelastic properties of the rubber compound (Tan delta at 0° C. and 60° C.) were measured via Dynamic Mechanical Analysis (DMA, Q-800, TA Instruments) of the cured tread rubber compounds using a tension film clamp, oscillation amplitude of 6 microns, 14 Hz frequency, and static force of 0.05N.
Molecular weights (Mn, Mw, Mz, and Mp) were measured using gel permeation chromatography (GPC, also called size exclusion chromatography or SEC) using the equipment and procedures as set out below and polystyrene standards.
p GPC Equipment: Agilent 1260 Series Degasser (Part Number G1322A), Isocratic Pump (Part Number G1310B), Autosampler (Part Number G1329B), Thermostatted Column Compartment (Part Number G1316A), Multiple Wavelength Detector (Part Number G1365C), and Refractive Index Detector (Part Number G1362A).
GPC Columns: 1× Agilent ResiPore 50×4.6 mm Guard Column (Part Number PL1513-1300) and 2× Agilent ResiPore 250×4.6 mm 3 um Particle Size Columns (Part Number PL1513-5300)
GPC Software: Cirrus 3.3, ChemStation B04.03 [52].
GPC Solvent: THF Stabilized with 250 ppm BHT.
GPC Flow Rate: 0.45 ml/min.
GPC Column Compartment Temperature: 40° C.
GPC Injection Volume: 5 μ.l
GPC Sample Preparation: Approximately 0.06 grams of sample was weighed into a vial, dissolved into 10 ml THF and then filtered through a 0.45 micron PTFE membrane.

All samples were run the same day as prepared.

GPC Calibration: Agilent EasiCal PS-2 Polystyrene Standards (Part Number PL2010-0605).

Room temperature viscosity was measured using a Brookfield DV-I+ digital viscometer with the small sample adapter. The sample was first placed in the cup of the viscometer and then the cup was positioned in the adapter sleeve by pushing it up from the bottom. Then the spindle 21 is correctly positioned in the sample. Next, the temperature bath was set to the desired temperature, i.e. 25° C. or 55° C.±0.1° C. The viscometer was run for a minimum of 30 minutes at a speed between 0.1 to 10 RPM such that the reading is between 20% and 80% of the viscometer range. After 30 minutes, the temperature readout was checked to determine that the temperature is correct and within ±0.1° C. in order for the viscosity reading to be valid. If the temperature is correct, the viscosity was recorded. If temperature was not correct, it was rechecked after 15 minutes.

### Example 1. Preparation of Liquid Oligomers

The following liquid resins as additives were prepared as follows.

Sample 1 was prepared in a glass reactor equipped with a stirrer and isothermal jacket that was purged with nitrogen for 30 minutes and throughout the course of the reaction. 45 g of a hexane/toluene mixture (80% hexane) was added to the reactor and warmed to 35° C. A monomer/solvent feed of 198.8 g with 50.2% actives was formulated using 51.0 g of piperylene rich C5 feed (about 60% dienes), 86.0 g inert C5 feed, 17.8 g isoamylenes, and 45.2 g a-methylstyrene, with 0.08 mL of isopropyl alcohol added as cocatalyst. A peristaltic pump was prepared to drip monomer feed into the reactor at 2 mL/min over 130 min. 1.70 g boron trifluoride diethyl etherate catalyst was prepared in a syringe pump that fed into the reactor at 0.7 mL/hr. Both monomer and catalyst feed were started simultaneously and fed for 130 min, while holding a constant reactor temperature. After the additions were completed, the reactor contents were stirred another 45 minutes to ensure complete use of catalyst. The reaction was quenched by the addition of 50 g aqueous 2-propanol (25%). After agitating the mixture, the layers were allowed to separate over 30 minutes. The organic phase was isolated and washed twice more with the same amount of 25% 2-propanol. 1000 ppm of Irganox® 1010 (BASF) as an antioxidant was added to the organic phase in a 3-neck round bottom flask. The mixture was purged with nitrogen while being heated to 160° C. during which time volatile organics were collected. Once at temperature, the nitrogen was replaced with steam. Oligomeric product and steam condensate were collected until approximately one gram of steam distillate per gram resin product was obtained. Yield was 79.0 g, or 79.2% of expected 99.8 g of resin, and was characterized via number average molecular weight (Mn: 460) and Tg (−16.9° C.) using GPC and DSC, respectively.

The compositions and the initiator, as well and Mn and Tg are shown in Table 1.
WTSTS is Wingtack STS from Resin Solutions.
WT10 is Wingtack® 10 from Resin Solutions, LLC
WT86 is Wingtack 86 from Resin Solutions, LLC
Sample 1 is a representative sample of the invention.
Sample 2 is a comparative sample of the invention, prepared in the same manner as
Sample 1, but having higher molecular weight and Tg (Table 1).
Buna® CB 24 (ARLANXEO) is a neodymium catalyzed butadiene rubber.
Buna® VSL 4526-2 HM (ARLANXEO) is a solution Styrene-Butadiene. Rubber containing 26% of bounded styrene.
Zeosil® 1165 MP (Solvay) is a precipitated amorphous silica with the average primary particle size of 20 nm and BET specific surface area of 153 m²/g).
Xiameter® OFS-6945 (Dow Silicones Corp.) is a sulfido tri ethoxy silane coupling agent extended on carbon black (50 wt % active).
6PPD is N-(1,3-dimethylbutyl)-N′-phenyl-p-phenylenediamine antioxidant
TBBS is N-tert-butyl-2-benzothiazolimesulfonamide is an aftereffect accelerator
DPG is Diphenyl Guanidine accelerator
TbzTd is tetrabenzylthiuram disulfide accelerator

| | | | | Sample 1 | Sample 2 |
|---|---|---|---|---|---|
| Monomer/properties | WT10 | WTSTS | WT86 | (invention) | (comparative) |
| % Aliphatic | | | | | |
| Monomer | | | | | |
| % Aromatic | 0 | 25 | 30 | 45 | 45 |
| Monomer | | | | | |
| Mn, g/mol | 370 | 1000 | 650 | 411 | 1032 |
| Tg, ° C. | −31 | 44 | 42 | −14 | 43 |

### Example 2: Tire Compositions

The samples listed in Table 1 were mixed in a model silica tire tread compounds, shown in Table 2. Resins replaced the TDAE process oil at 20 phr. In the results section below, the compounds will be identified by the resin acronym (from Table1). All compounds were prepared in three stages in an internal mixer (Brabender Prep-Mixer, 420 ml capacity, with Banbury blades and Intelli-Torque Plastic-Corder 7.5 hp drive), with the following conditions:
Stage 1. 100° C., 50 RPM
Stage 2. 100° C., 50 RPM
Stage. 60° C., 35 RPM.

The compounds were calendared in between mixing stages using a lab scale two roll mill (Reliable Rubber and Plastic Machinery, 6″×13″ variable speed drive, Model 5025). Blends were removed and allowed to cool overnight prior to curing and analysis. All tread rubber model compounds were cured and molded in a heated press at 160° C. for 20 minutes.

TABLE 2
Rubber compositions

| | Comp | Comp | Comp | Comp | Comp | Comp | Ex. A | Comp | Comp |
|---|---|---|---|---|---|---|---|---|---|
| | A | B | C | D | E | F | WT10 | G | H |
| | TDAE | R100 | W85 | WT10 | WTSTS | WT86 | AMS | WT45 | TDAE |
| Buna ® CB24 | 25 | 25 | 25 | 25 | 25 | 25 | 25 | 25 | 25 |
| Buna(D) | 103.1 | 103.1 | 103.1 | 103.1 | 103.1 | 103.1 | 103.1 | 103.1 | 103.1 |
| VSL4626-2HM | | | | | | | | | |
| Zeosil ® 1165 MP | 90.0 | 90.0 | 90.0 | 90.0 | 90.0 | 90.0 | 90.0 | 90.0 | 90.0 |
| Xiameter ® OFS- | 14.0 | 14.0 | 14.0 | 14.0 | 14.0 | 14.0 | 14.0 | 14.0 | 14.0 |
| 6945 | | | | | | | | | |
| TDAE | 20 | — | — | — | — | — | — | — | 20 |
| WT10 | — | — | — | 20 | — | — | — | — | — |
| WTSTS | — | — | — | — | 20 | — | — | — | — |
| WT86 | — | — | — | — | — | 20 | — | — | — |
| Sample 1 | — | — | — | — | — | — | 20 | — | — |
| Sample 2 | — | — | — | — | — | — | — | 20 | — |
| Stearic acid | 2.3 | 2.3 | 2.3 | 2.3 | 2.3 | 2.3 | 2.3 | 2.3 | 2.3 |
| 6PPD | 2.0 | 2.0 | 2.0 | 2.0 | 2.0 | 2.0 | 2.0 | 2.0 | 2.0 |
| TBBS | 1.7 | 1.7 | 1.7 | 1.7 | 1.7 | 1.7 | 1.7 | 1.7 | 1.7 |
| Sulfur | 1.3 | 1.3 | 1.3 | 1.3 | 1.3 | 1.3 | 1.3 | 1.3 | 1.3 |
| DPG | 2.0 | 2.0 | 2.0 | 2.0 | 2.0 | 2.0 | 2.0 | 2.0 | 2.0 |
| Zinc oxide | 2.3 | 2.3 | 2.3 | 2.3 | 2.3 | 2.3 | 2.3 | 2.3 | 2.3 |
| TBzTD | 0.5 | 0.5 | 0.5 | 0.5 | 0.5 | 0.5 | 0.5 | 0.5 | 0.5 |

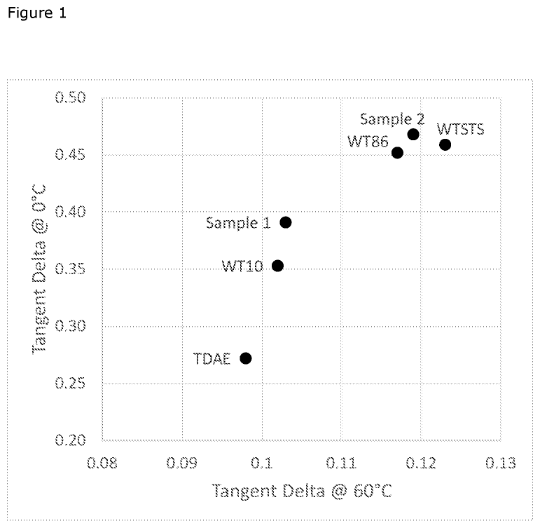

The tan delta at 0° C., which correlates to traction, is shown plotted as a function of tan delta at 60° C., which correlates to rolling resistance, in FIG. 1. As shown in FIG. 1, the low molecular weight liquid oligomers according to the invention provide similar or improved (higher) traction compared to the comparative R100 additive in a cured rubber composition while also provide significantly improved (lower) rolling resistance. In particular, the WT10 and Sample 1 samples exhibited significantly improved rolling resistance and traction compared to the comparative samples. These samples also provide similar rolling resistance to the oil (TDAE) and very much better traction.

What is claimed is:

1. A curable rubber composition comprising:
a) a liquid oligomer having a number average molecular weight Mn 600 g/mol or less, and a glass transition temperature Tg of −10° C. or less as measured by Differential Scanning calorimetry (DSC), comprising, as polymerized monomers, at least one diene, at least one branched mono-olefin, and optionally at least one vinyl aromatic monomer;
b) at least one polymeric diene-based elastomer having a number average molecular weight of 100,000 g/mol or higher;
c) at least one filler comprising at least one of silica, carbon black, or a combination thereof and;
d) optionally a curative system capable of curing the curable rubber composition when heated.

2. The curable rubber composition of claim 1, wherein the at least one diene comprises C5 dienes, dimers thereof, or combinations thereof.

3. The curable rubber composition of claim 1, wherein the at least one diene comprises at least one of piperylene, isoprene, or combinations thereof.

4. The curable rubber composition of claim 1, wherein the at least one branched mono-olefin comprises at least one of isoamylene, isobutylene, or combinations thereof.

5. The curable rubber composition of claim 1, wherein the liquid oligomer comprises from 0 to 50 wt % of the vinyl aromatic monomer, by weight of the liquid oligomer.

6. The curable rubber composition of claim 1, wherein the vinyl aromatic monomer comprises at least one of styrene, alpha-methyl styrene, meta-methyl styrene, para-methyl styrene, vinyltoluene, para-t-butylstyrene, methoxystyrenes, chlorostyrenes, vinylmesitylene, divinylbenzene, vinylnaphthalene, or combinations thereof.

7. The curable rubber composition of claim 1, wherein the Mn of the liquid oligomer is 200 to 600 g/mol.

8. The curable rubber composition of claim 1, wherein the Tg of the liquid oligomer is −70 to −10° C.

9. The curable rubber composition of claim 1, wherein the liquid oligomer further comprises, as polymerized monomers, at least one myrcene, farnesene, or combination thereof.

10. The composition of claim 1, wherein the liquid oligomer comprises, as polymerized monomers, by weight of the liquid oligomer:
from 40 to 50 wt % of alpha-methyl styrene;
from 25 to 60 wt % of at least one C5 diene comprising at least one of piperylene, isoprene, pentadiene, dicyclopentadiene, or combinations thereof;
from 20 to 50 wt % of at least one branched olefin comprising at least one of isoamylene, isobutylene, or combinations thereof;
optionally from 0 to 60 wt % at least one of myrcene, farnesene, divinylbenzene, or combinations thereof;
and wherein the liquid oligomer has a Tg from −70 to −10° C. and a number average molecular weight Mn from 300 to 600 g/mol.

11. The curable rubber composition of claim 1, wherein the polymeric diene-based elastomer comprises at least one of polybutadienes, polyisoprenes, natural rubber, copolymers of butadiene and vinyl aromatic monomers, poly (styrene-co-butadiene), copolymers of isoprene and vinyl aromatic monomers, styrene-isoprene-butadiene rubber (SIBR), styrene-isoprene rubber (SIR), isoprene-butadiene rubber (IBR), natural rubber, polybutadiene, including cis 1,4-polybutadiene), polyisoprene, including cis-1,4-polyisoprene, ethylene/propylene/diene terpolymers (EPDM), and combinations thereof.

12. The curable rubber composition of claim 1, wherein the polymeric diene-based elastomer further comprises at least one functional group configured to interact with the filler through covalent or ionic bonds.

13. The curable rubber composition of claim 12, the polymeric diene-based elastomer further comprising an elastomer chain comprising two end portions and a middle portion disposed between the two end portions and
wherein the at least one functional group is located at either or both of the two end portions, the middle portion, or a combination thereof.

14. The curable rubber composition of claim 13, wherein the at least one functional group is added to the middle portion of the elastomer chain through a coupling reaction or a grafting reaction.

15. The curable rubber composition of claim 13, wherein the at least one functional group is one or more of an alkoxysilane or an amine.

16. The curable rubber composition of claim 1, further comprising at least one silane coupling agent, wherein the silane group is represented by the following formula: —Si(OR)₃, where each R is independently a C1-C6 alkyl group or an aryl group.

17. The curable rubber composition of claim 1, wherein the liquid oligomer is produced via Lewis acid-initiated polymerization.

18. The curable rubber composition of claim 17, wherein the Lewis acid comprises at least one of AlCl₃, BF₃ or BF₃ etherate.

19. The curable rubber composition of claim 1, wherein the curative system comprises at least one of elemental sulfur, organosulfur compounds, or combinations thereof.

20. The curable rubber composition of claim 19, wherein the curative system additionally comprises an accelerator, a curing aid, an activator or a combination thereof.

21. A cured rubber composition obtained by curing the curable rubber composition of claim 1.

22. A tire tread, comprising the cured rubber composition of claim 21.

23. A tire, comprising the tire tread of claim 22.

24. A method of making a tire tread, comprising molding and curing the composition of claim 1.

25. A method of preparing a liquid oligomer comprising:
combining:
at least one diene,
at least one branched mono-olefin,
optionally at least one vinyl aromatic monomer, and
at least one initiator comprising a Lewis acid,
to form a reaction mixture;
polymerizing the reaction mixture at a temperature and time effective to form the liquid oligomer;
wherein the liquid oligomer comprises, as polymerized monomers,
the at least one diene,
the at least one branched mono-olefin,
and optionally the at least one vinyl aromatic monomer;
and has a number average molecular weight Mn of 600 g/mol or less, and a glass transition temperature Tg of −10° C. or less as measured by differential scanning calorimetry (DSC).

26. The method of claim 25, wherein the temperature is from 5 to 45° C.

27. The method of claim 25, wherein the Lewis acid comprises at least one of AlCl₃, BF₃ or BF₃ etherate.

28. The method of claim 25, wherein the initiator further comprises a protic source.

29. The method of claim 25, wherein the at least one diene comprises C5 dienes, or combinations thereof.

30. The method of claim 25, wherein the at least one diene comprises at least one of piperylene, isoprene, pentadiene, dicyclopentadiene, or combinations thereof.

31. The method of claim 25, wherein the at least one branched mono-olefin comprises at least one of isoamylene, isobutylene, or combinations thereof.

32. The method of claim 25, wherein the liquid oligomer comprises from 0 wt % to 50 wt % of the vinyl aromatic monomer, by weight of the liquid oligomer.

33. The method of claim 25, wherein the vinyl aromatic monomer comprises at least one of styrene, alpha-methyl styrene, meta-methyl styrene, para-methyl styrene, vinyltoluene, para-t-butylstyrene, methoxystyrenes, chlorostyrenes, vinylmesitylene, divinylbenzene, vinylnaphthalene, or combinations thereof.

34. The method of claim 25, wherein the Mn of the liquid oligomer is 200 to 600 g/mol.

35. The method of claim 25, wherein the Tg of the liquid oligomer is −70 to −10° C.

36. The method of claim 25, wherein the combining further comprises combining at least one of myrcene, farnesene, or combinations thereof.

37. The method of claim 25, wherein the reaction mixture comprises:
from 40 to 50 wt % of alpha-methyl styrene;
from 25 to 60 wt % of at least one C5 diene comprising at least one of piperylene, isoprene, dicyclopentadiene, or combinations thereof;
from 20 to 50 wt % of at least one branched olefin comprising at least one of isoamylene, isobutylene, or combinations thereof;
optionally from 0 to 60 wt % at least one of, myrcene, farnesene, divinylbenzene, or combinations thereof;
and wherein the liquid oligomer has a Tg from −70 to −10° C. and a number average molecular weight Mn from 300 to 600 g/mol.
